(19) United States
(12) Patent Application Publication
(10) Pub. No.: US 2025/0017948 A1
(43) Pub. Date: Jan. 16, 2025

(54) CANNABINOID COMPOSITIONS FOR GASTROESOPHAGEAL DISORDERS

(71) Applicants: THE UNITED STATES GOVERNMENT AS REPRESENTED BY THE DEPARTMENT OF VETERANS AFFAIRS, Washington, DC (US); THE BRIGHAM AND WOMEN'S HOSPITAL, Boston, MA (US)

(72) Inventors: Aaron J. Goldman, Melrose, MA (US); Gabriel Gonzalez, Needham, MA (US); Natasha Y. Frank, Boston, MA (US)

(21) Appl. No.: 18/702,146

(22) PCT Filed: Oct. 19, 2022
(86) PCT No.: PCT/US2022/047125

§ 371 Date: Apr. 17, 2024

Related U.S. Application Data
(60) Provisional application No. 63/257,305, filed on Oct. 19, 2021.

(51) Int. Cl. A61K 31/00; A61K 31/015; A61K 31/045; A61K 47/36

(52) U.S. Cl. CPC A61K 31/658; A61K 31/015; A61K 31/045; A61K 47/36

(57) ABSTRACT

Compositions comprising a cannabinoid and other compounds, such as terpenes, and methods of using such compositions. The disclosed compositions can be useful in, for example, treating and preventing esophageal adenocarcinoma and related diseases such as esophageal dysplasia, esophageal metaplasia, Barrett's Esophagus, gastroesophageal reflux disorder (GERD), or conditions associated with acid-biliary reflux.

### CROSS-REFERENCE TO RELATED APPLICATIONS

This application claims the benefit of U.S. Provisional Application No. 63/257,305, filed on Oct. 19, 2021, which is incorporated herein by reference in its entirety.

### STATEMENT REGARDING FEDERALLY SPONSORED RESEARCH

This invention was made with U.S. Government support under Contract No. K01CA226375 awarded by the National Institutes of Health (NIH) and the National Cancer Institute (NCI). The government has certain rights in the invention.

### BACKGROUND

Esophageal adenocarcinoma (EAC) is a highly aggressive malignancy associated with Barrett's Esophagus (BE), dysplasia and metaplasia, and conditions affiliated with chronic exposure to acid-biliary reflux and gastroesophageal reflux disorder (GERD). Bile acids, such as deoxycholic acid (DCA), enter the esophagus during an episode of reflux, and are thought to promote cancer development. Patients with GERD and BE show high concentrations of DCA in their refluxate, which has cytotoxic effects and can induce DNA damage through a process that involves induction of reactive oxygen species (ROS) and disruption of lysosomal integrity that can drive ionic perturbations. The resulting ROS insult and oxidative damage drive genotoxicity and DNA double-strand breaks (DSBs). There remain few effective courses of therapy to suppress malignant transformation and development of EAC. This highlights the need for development of novel therapeutic strategies to counteract or prevent the carcinogenic effects of acid-biliary reflux.

### SUMMARY

In one aspect, this disclosure relates to a pharmaceutical composition of a cannabinoid represented by Formula (I):

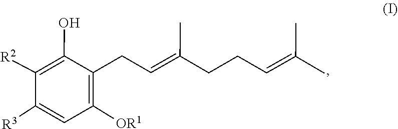

where R¹ can be hydrogen or methyl, R² can be hydrogen or —COOH, R³ can be a C3-C5 alkyl, or a salt thereof, together with a compound presented by formula II:

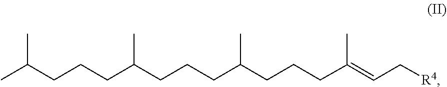

where R⁴ can be a halide or —OR⁵, where R⁵, when present, can be hydrogen or —COR⁶, where R⁶, when present, can be a C1-C18 alkyl or an aryl.

Alternatively, the compound of Formula (1) can be present together with a terpene selected from (E)-β-caryophyllene, (Z)-β-caryophyllene, caryophyllene oxide, α-humulene, myrcene, limonene, linalool, or pinene. The cannabinoid and the compound represented by Formula (II), or the terpene, can be present at a non-naturally occurring molar ratio. In addition, the compound represented by Formula (II), or the terpene, can be present at a molar amount that exceeds the molar amount of the cannabinoid.

In a further aspect, this disclosure relates to a method of treating or preventing esophageal adenocarcinoma in a subject, the method comprising administering to the subject an effective amount of the composition of the cannabinoid of Formula (I) together with the compound represented by Formula (II), or the terpene.

In a further aspect, this disclosure relates to a method comprising administering to a subject that has or has been diagnosed with esophageal dysplasia, esophageal metaplasia, Barrett's Esophagus, gastroesophageal reflux disorder (GERD), or a condition associated with acid-biliary reflux, an effective amount of the composition of the cannabinoid of Formula (I) together with the compound represented by Formula (II), or the terpene.

In a further aspect, this disclosure relates to a method of counteracting deoxycholic acid (DCA)-mediated mitochondrial or DNA damage in an esophageal cell, the method comprising contacting the esophageal cell with an effective amount of a composition comprising the cannabinoid of Formula (I) together with the compound represented by Formula (II), or the terpene.

In a further aspect, this disclosure relates to a method of inducing mitochondrial membrane depolarization in an esophageal epithelial cell, the method comprising contacting the cell with an effective amount of a composition comprising the cannabinoid of Formula (I) together with the compound represented by Formula (II), or the terpene.

In a further aspect, this disclosure relates to a method of decreasing DNA damage in an esophageal epithelial cell, the method comprising contacting the cell with an effective amount of a composition comprising the cannabinoid of Formula (I) together with the compound represented by Formula (II), or the terpene.

In a further aspect, this disclosure relates to a method of reducing cell proliferation of an esophageal epithelial cell exposed to deoxycholic acid (DCA), the method comprising contacting the cell with an effective amount of a composition comprising the cannabinoid of Formula (I) together with the compound represented by Formula (II), or the terpene.

In a further aspect, this disclosure relates to a method of reducing or preventing low pH or bile-induced reactive oxygen species (ROS) in an esophageal cell, the method comprising contacting the esophageal cell with an effective amount of a composition comprising the cannabinoid of Formula (I) together with the compound represented by Formula (II), or the terpene.

In another aspect, disclosed are the cannabinoid compositions together as a hydrogel formed from an alginate and an amine such as a hydrophobic amine.

Still other objects and advantages of the present disclosure will become readily apparent by those skilled in the art from the following detailed description, which is shown and described by reference to preferred aspects, simply by way of illustration of the best mode. As will be realized, the disclosure is capable of other and different aspects, and its several details are capable of modifications in various respects, without departing from the disclosure. Accordingly, the description is to be regarded as illustrative in nature and not as restrictive.

### BRIEF DESCRIPTION OF THE DRAWINGS

The accompanying drawings, which are incorporated in and constitute part of this specification and together with the description, serve to explain the principles of the disclosure.

FIG. 1A shows plots showing membrane depolarization of HET1A measured by red/green fluorescent changes (JC-1) after acute exposure to DCA (100-500 mM).

FIG. 1B shows H2A.X and ATM DNA damage response as measured in HET1A cells treated with a range of cannabinoids and terpenes at 1:5 ratio, respectively, prior to and during exposure with 100 mM DCA. Vehicle control cohort indicates the level of ATM and H2A.X activity following exposure to DCA alone.

FIG. 1C shows mitochondrial membrane depolarization in HET1A treated with the indicated combination of cannabigerol (CBG) and the terpene phytol (Phy) prior to and during exposure with 100 mM DCA.

FIG. 1D is a bar graph showing the ratio of JC-1 red/green fluorescence in HET1A cells treated with CBG/Phytol at 1:5 ratio prior to and during exposure with 300 mM DCA. Values are compared to the vehicle control cohort.

FIGS. 1A-E show that Cannabigerol and phytol combined at a ratio of 1:5 optimally reverse DCA stress-induced mitochondrial depolarization and DNA damage response in surviving squamous epithelial cells. FIG. 1E is a bar graph showing DNA double strand breaks measured in HET1A cells treated with the indicated combination of CBG and Phytol at 1:5 ratio, respectively, prior to and during exposure with 100 mM DCA.

FIG. 2 is a cell viability analysis of immortalized squamous epithelial cells (HET1A) following acute exposure to deoxycholic acid at indicated doses. Data were evaluated by XTT assay and are expressed as a % of vehicle control cohort.

FIG. 3 is a bar graph showing the results of human esophageal epithelial cells (HEsEpiC) analyzed for DNA damage following treatment with DCA (100 mM)+/−the indicated combination of cannabinoid and terpene at a ratio of 1:5.

FIG. 4A is a plot showing cell viability in HET1a cells treated with or without 100 mM DCA for 24 hours and with or without CBG and/or Phytol for 72 hours.

FIG. 4B is a plot representing the percentage of apoptotic cells with or without CBG/Phytol pre-treatment and treated with DCA ranging from 0 to 500 mM. FIGS. 4A-4B show that pre-treatment and maintenance treatment of CBG and phytol combined at a ratio of 1:5 prevented hallmarks of apoptosis resistance in squamous epithelial cells following exposure to DCA at toxic doses.

FIG. 5A shows images that illustrate the copy number events acquired by control and treated cells in three weeks, relative to day 0 cells throughout chromosomes.

FIG. 5B is a bar graph showing the total number of copy-number change events throughout the genome. FIGS. 5A-B show that CBG+Phytol (1:5) reduces DCA-induced genomic instability in squamous epithelial cells. HET1A cells were cultured either with vehicle, 100 μM DCA, CBG/Phytol admixture or combination of DCA and CBG/Phytol admixture for 14 days. DNA from these and parental (Day 0 cells) extracted and analyzed using PMDA arrays (Affymetrix). Genomic instability in cultured cells was assessed by identifying new copy number events (both deletions and amplifications), using genome of “Day 0” cells as baseline.

FIG. 6A is an experimental design schematic. Non-dysplastic BE cells (CP-A) were treated with a vehicle control or cannabinoids in the presence or absence of terpenes for 12 hours followed by an acute (15 minutes) exposure to physiologically relevant low pH and bile acid cocktail (see Examples). The cells were either immediately analyzed for ROS via fluorescent detection of CM-DCFDA or recovered for 24 hours and analyzed for DNA damage, via γ-H2AX.

FIG. 6B shows reactive oxygen species (ROS) measured by CM-DCFDA fluorescence in CP-A cells following acute exposure to GER. Data analyzed by flow cytometry and expressed as a % increase vs. vehicle control.

FIG. 6C is a cell viability analysis of CP-A cells following exposure to cannabinoids for 24 h.

FIG. 6D is a heat map illustrating a matrix of ROS induced by GER in CP-A cells that were pre-treated with the indicated conditions and cannabinoid and terpene combinations (at a 1:5 ratio). Data are expressed as the % change from GER alone.

FIG. 6E are γ-H2AX fluorescence plots used to determine DNA damage via flow cytometry in CP-A cells following exposure to GER.

FIG. 6F is a bar graph showing CM-DCFDA fluorescence to compare the effect of GER with anti-oxidants (catalase), ursodeoxycholic acid or the cannabinoid/terpene combination of CBG and b-car at 1:5 ratio. FIGS. 6A-F show that CBG and β-caryophyllene combined at a ratio of 1:5 optimally reversed acid-containing gastroesophageal refluxate (GER) in metaplastic Barrett's esophagus cells.

FIGS. 7A-B are volcano plots showing the results of the GSE database (GSE 1420) query. The database was queried for significantly upregulated or downregulated genes (p-adj <0.05) between normal vs. BE (A) or normal vs. BE-associated adenocarcinoma (B). G-protein coupled receptors were identified in each data set and annotated in the plots. FIGS. 7A-7B show that Barrett's esophagus microarray linked Barrett's esophagus (BE) and BE-associated adenocarcinoma with orphan GPCR.

FIGS. 8A-D are confocal microscopy images showing the expression of CB1 in human esophageal mucosa and esophageal adenocarcinoma. FIG. 8A shows a representative image of stratified squamous epithelium; inset presented to show expression of CB1 in basal epithelial layer. FIG. 8B shows an image of ductal epithelium of submucosal gland (d) and acini of the submucosal gland (a). FIGS. 8C-D show representative images of esophageal adenocarcinoma. EPCAM was used to detect cancer cells.

FIG. 8E is an H-score plot representing semiquantitative intensity scoring analysis of CB1 expression in normal stratified squamous epithelial cells and esophageal adenocarcinoma.

FIG. 9 is a schematic showing the formation of a sodium alginate derivative comprising a defined ratio of phytol and cannabigerol.

FIG. 10 is a brightfield microscope image of sodium alginate-drug formulation.

### DETAILED DESCRIPTION

The present disclosure can be understood more readily by reference to the following detailed description of the invention and Examples.

Disclosed are components that can be used to perform the disclosed methods. These and other components are disclosed, and it is understood that when combinations, subsets, interactions, groups, etc. of these components are disclosed that while specific reference of each various individual and collective combinations and permutation of these may not be explicitly disclosed, each is specifically contemplated and described, for all methods and products. This applies to all aspects of this application including, but not limited to, steps in disclosed methods. Thus, if there are a variety of additional steps that can be performed it is understood that each of these additional steps can be performed with any specific embodiment or combination of embodiments of the disclosed methods.

While aspects of this disclosure can be described and claimed in a particular statutory class, this is for convenience only and one of skill in the art will understand that each aspect of this disclosure can be described and claimed in any statutory class. Unless otherwise expressly stated, it is in no way intended that any method or aspect set forth herein be construed as requiring that its steps be performed in a specific order. Accordingly, where a method claim does not specifically state in the claims or description that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

Throughout this application, various publications are referenced. The disclosures of these publications in their entireties are incorporated by reference into this application in order to more fully describe the state of the art to which this pertains. The references disclosed are also individually and specifically incorporated by reference for the material contained in them that is discussed in the sentence in which the reference is relied upon. Nothing is to be construed as an admission that the present application is not entitled to antedate such publication by virtue of prior invention. Further, stated publication dates may be different from actual publication dates, which can require independent confirmation.

### A. DEFINITIONS

Listed below are definitions of various terms. These definitions apply to the terms as they are used throughout this specification, unless otherwise limited in specific instances, either individually or as part of a larger group.

The term “about” refers to the stated value plus or minus 10%.

“Alkyl” means a branched or unbranched saturated hydrocarbon. The alkyl group can also be substituted or unsubstituted. For example, the alkyl group can be substituted with one or more groups including, but not limited to amino, ether, halide, hydroxy, nitro, silyl, among others. Alkyl can also be cyclic or acyclic. Examples of “C3-C5 alkyl” include, but are not limited to, n-propyl, isopropyl, n-butyl, isobutyl, s-butyl, t-butyl, n-pentyl, isopentyl, s-pentyl, and neopentyl. Examples of “C1-C18” alkyl include, but are not limited to, methyl, ethyl, n-propyl, isopropyl, n-butyl, isobutyl, s-butyl, t-butyl, n-pentyl, isopentyl, s-pentyl, neopentyl, hexyl, heptyl, octyl, nonyl, decyl, dodecyl, tetradecyl, hexadecyl, eicosyl, tetracosyl, and the like.

“Aryl” means a group that contains any carbon-based aromatic group including, but not limited to, benzene, naphthalene, phenyl, biphenyl, anthracene, and the like. The aryl group can be substituted or unsubstituted. The aryl group can be substituted with one or more groups including, but not limited to, alkyl, cycloalkyl, alkoxy, alkenyl, cycloalkenyl, alkynyl, cycloalkynyl, aryl, heteroaryl, aldehyde, —NH₂, carboxylic acid, ester, ether, halide, hydroxy, ketone, azide, nitro, silyl, sulfo-oxo, or thiol as described herein. The term “biaryl” is a specific type of aryl group and is included in the definition of “aryl.” In addition, the aryl group can be a single ring structure or comprise multiple ring structures that are either fused ring structures or attached via one or more bridging groups such as a carbon-carbon bond. For example, biaryl can be two aryl groups that are bound together via a fused ring structure, as in naphthalene, or are attached via one or more carbon-carbon bonds, as in biphenyl.

The terms “halo,” “halogen,” or “halide” can be used interchangeably and refer to F, Cl, Br, or I.

“Terpene” refers to a class of unsaturated compounds (i.e., a compound including at least one carbon-carbon double bond) that include the general formula (C₅H₈)ₙ, where n is an integer. Terpenes can be classified by the number of carbons: monoterpenes (C₁₀), sesquiterpenes (C₁₅), diterpernes (C₂₀), and the like, and thus “terpene” encompasses hemiterpenes, monoterpenes, sesquiterpenes, diterpenes, sesterterpenes, triterpenes, tetraterpenes and polyterpenes. The diene moiety of a terpene may have any stereochemistry (e.g., cis or trans) and may be part of a longer (in some cases, conjugated) segment of a terpene, e.g., a conjugated diene moiety may be part of a conjugated triene moiety. A terpene may contain a conjugated diene at a terminal position (e.g., myrcene, famesene) or a conjugated diene may be at an internal position (e.g., isodehydrosqualene or isosqualane). Some non-limiting examples of terpenes include isoprene, myrcene, (E)-β-caryophyllene, (Z)-β-caryophyllene, caryophyllene oxide, α-humulene, limonene, linalool, and pinene.

The term “cannabinoid” refers to a class of chemical compounds capable of interacting with any mammalian cannabinoid receptor, for example the human CB₁ or CB₂ receptor. The term encompasses naturally-occurring cannabinoids (e.g., phytocannabinoids found in the cannabis plant), synthetic cannabinoids, cannabinoid mimetics, as well as salts, precursors, and metabolites of cannabinoids.

The term “molar ratio” refers to the moles of one component divided by the moles of another component. For example, if the molar ratio of cannabinoid to terpene is 1:5, then for every mole of cannabinoid, there are five moles of terpene.

“Substantially free of” of a stated component refers to a composition having less than about 10% by weight of the stated component, e.g., less than about 5%, less than about 1%, less than about 0.5%, less than about 0.1%, less than about 0.05%, or less than about 0.010% by weight of the stated material, based on the total weight of the composition. “Free of” means the composition has no measurable amount of the stated component.

The term “acceptable, non-naturally occurring salt” refers to refers to a salt formed by the addition of an acid or base to a compound. The phrase “acceptable” refers to a material that is technically acceptable for pharmaceutical use and that does not negatively interact with the active ingredient. These salts include, but are not limited to, those derived from organic and inorganic acids such as acetic acid, lactic acid, citric acid, cinnamic acid, tartaric acid, succinic acid, fumaric acid, maleic acid, malonic acid, mandelic acid, malic acid, oxalic acid, propionic acid, hydrochloric acid, hydrobromic acid, phosphoric acid, nitric acid, sulfuric acid, glycolic acid, pyruvic acid, methanesulfonic acid, ethanesulfonic acid, Toluenesulfonic acid, salicylic acid, benzoic acid and similarly known acids.

The term “non-naturally occurring carrier” refers to sterile aqueous or nonaqueous solutions, dispersions, suspensions or emulsions, as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol and the like), carboxymethylcellulose and suitable mixtures thereof, vegetable oils (such as olive oil) and injectable organic esters such as ethyl oleate.

The terms “treating” or “treatment” refer to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder. In various aspects, the term covers any treatment of a subject, including a mammal (e.g., a human), and includes: (i) preventing the disease from occurring in a subject that can be predisposed to the disease but has not yet been diagnosed as having it; (ii) inhibiting the disease, i.e., arresting its development; or (iii) relieving the disease, i.e., causing regression of the disease.

The terms “prevent” or “preventing” refers to precluding, averting, obviating, forestalling, stopping, or hindering something from happening, especially by advance action. It is understood that where reduce, inhibit or prevent are used, unless specifically indicated otherwise, the use of the other two words is also expressly disclosed.

The term “esophageal adenocarcinoma” (ECA) refers to a sub-type of esophageal carcinoma characterized by neoplasia of epithelial tissue that has glandular origin, glandular characteristics, or both, and is typically present in the lower third of the esophagus.

The term “subject” can be any subject, including a mammalian subject such as a human.

The terms “administering” and “administration” refer to any method of providing a pharmaceutical preparation to a subject. Such methods are well known to those skilled in the art and include, but are not limited to, oral administration, transdermal administration, administration by inhalation, nasal administration, topical administration, among other routes of administration.

The term “effective amount” refers to an amount that is sufficient to achieve the desired result or to have an effect on an undesired condition. For example, a “therapeutically effective amount” refers to an amount that is sufficient to achieve the desired therapeutic result or to have an effect on undesired symptoms, but is generally insufficient to cause adverse side effects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors well known in the medical arts. For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. Dosage can vary, and can be administered in one or more dose administrations daily, for one or several days. Guidance can be found in the literature for appropriate dosages. In further various aspects, a preparation can be administered in a “prophylactically effective amount”; that is, an amount effective for prevention of a disease or condition.

The term “esophageal dysplasia” refers to abnormal cell growth or proliferation of esophageal cells.

The term “esophageal metaplasia” refers to uncontrolled cell growth in which one type of adult or fully differentiated cell, specifically intestinal or Goblet cells, substitutes for another type of adult cell, specifically squamous esophageal cells.

The term “Barrett's esophagus” refers to an abnormal change (metaplasia) in the cells of the lower portion of the esophagus. Barrett's esophagus can be characterized by the finding of intestinal metaplasia in the esophagus.

The term “Gastrointestinal reflux disorder” or “GERD” refers to the incidence and symptoms of conditions caused by the reflux of the stomach contents into the esophagus. The term includes all forms and manifestations of GERD including, but not limited to, erosive and non-erosive GERD, heartburn and other symptoms associated with GERD.

A “condition associated with acid biliary reflux” refers to the incidence of and symptoms of conditions caused by the reflux of bile into the esophagus. Specific conditions associated with acid biliary reflux include for example a variety of conditions discussed in context with the disclosed compositions and methods.

The term “esophageal cell” refers to refers to cells obtained from esophageal tissue. This may be a heterogeneous cell population comprising epithelial cells, smooth muscle cells, and any combination thereof. Esophageal cells can be obtained from esophageal biopsies or from whole esophageal tissue. Alternatively, esophageal cells can be obtained from esophageal tissue biopsies or in vitro culture of cell populations established from whole esophageal tissue. Esophageal cells are characterized by the expression of markers associated with epithelial cells, smooth muscle cells, and any combination thereof. The esophageal cell population may also be a purified cell population. Examples of esophageal cells include, but are not limited to, HET1a, Human Esophageal Epithelial Cells, and Human Primary Esophageal Epithelial Cells.

The term “mitochondrial membrane polarization” refers to the process in which the electrical potential difference between the compartments separated by the mitochondrial inner membrane is reduced from its steady state level.

The term “squamous esophageal cell” refers to epithelial cells that line the esophagus.

The term “cell proliferation” refers to an increase in the number of cells, which means that the rate of proliferation is faster than the rate of cell death (e.g., by apoptosis or necrosis). Cell proliferation occurs by propagation resulting in an increase in the size of a cell population, but a small portion of that proliferation may be due to an increase in the cell size or cytoplasmic volume of individual cells in certain circumstances. An agent that is described as inhibiting cell growth can do so by inhibiting growth or stimulating cell death, or both, such that the equilibrium between the two opposing processes is altered.

The terms “reactive oxygen species” or “ROS” refer to molecules or ions that contain oxygen ions, free radicals, peroxides, or combinations thereof. Reactive oxygen species may be organic or inorganic. Examples of reactive oxygen species include, but are not limited to, super oxides, free radicals, such as hydroxyl radicals and peroxyl radicals, peroxides, singlet oxygen, ozone, nitrogen monoxide, anions, such as hydroxyl anions and superoxide anions, hypochlorous acid, and peroxynitrites, as well as combinations of any such reactive oxygen species.

The term “low pH or bile-induced reactive oxygen species (ROS)” refers to reactive oxygen species (ROS) formed in conditions typically found in the caustic environment of gastroesophageal reflux (GER). These conditions may refer to in vitro or in vivo assay conditions that correspond to one or more conditions present in an in vivo environment. “Low pH” is defined as an acidic pH, that is, a pH lower than 7. GER is characterized by stomach acid or bile irritating the esophagus, causing a reduction of pH in esophageal cells. For example, if the microenvironment is characterized by a low pH, conditions that mimic the microenvironment include buffers or assay conditions that have a low pH. In some aspects, ROS are formed at a pH of about 4.5, in an environment comprised of multiple physiologically-relevant secondary bile acids such as glycocholate, taurocholate, glycodeoxycholate, glycochenodeoxycholate, and deoxycholate.

### B. CANNABINOID COMPOSITIONS

The endocannabinoid system (ECS) is mechanistically complex and diverse, mediating homeostasis throughout the body, influencing inflammation, metabolism, immunity and even cardiovascular health. While the discovery of endocannabinoids, such as anandamide (EAE), have led to greater interest in the endogenous modulation of the ECS, phytochemicals derived from the plant, Cannabis sativa, can mimic many of the same effects, which provide a wealth of ECS-modulating natural product pharmaceuticals. Although there are more than 100 cannabinoids and terpenes present in Cannabis sativa, there is limited information on their individual or combined medicinal qualities, particularly when isolated from the plant or synthesized and combined at quantities that do not occur naturally. Because it is not fully understood how cannabinoids and terpenes can be harnessed for therapeutic benefit, there exists a need in the art to establish the specific pathologies any one combination may effectively treat and the optimal ratios and combinations of phytochemicals that elicit these qualities. This need an others are satisfied by the following disclosure.

Cannabis is a genus of flowering plants that includes at least three species, Cannabis sativa, Cannabis indica, and Cannabis ruderalis. Cannabis plants produce a family of terpeno-phenolic compounds called cannabinoids. More than 100 cannabinoids have been identified from crude cannabis. Most cannabinoids exist in two forms, as acids and in neutral (decarboxylated) forms. The acid form is designated by an “A” at the end of its acronym, e.g., TCHA. Cannabinoids are synthesized in the plant as acid forms, and while some decarboxylation does occur in the plant, it increases significantly post-harvest, and the kinetics of decarboxylation increase at high temperatures. Decarboxylation can be achieved by thorough drying of the plant material followed by heating it or exposing it to light or alkaline conditions.

In one aspect, the disclosed compositions include a cannabinoid represented by Formula (I):

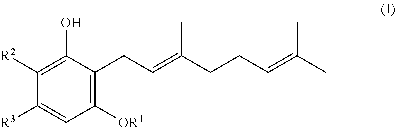

where R¹ is hydrogen or methyl; R² is hydrogen or —COOH; and R³ is C3-C5 alkyl; or a salt thereof. The C3-C5 alkyl can be n-propyl, isopropyl, n-butyl, isobutyl, s-butyl, t-butyl, n-pentyl, isopentyl, s-pentyl, neopentyl, among others. The C3-C5 alkyl can be branched or unbranched. The C3-C5 alkyl group can also be substituted or unsubstituted. In some aspects, the alkyl group can be substituted with one or more groups including amino, ether, halide, hydroxy, nitro, silyl, among others.

In a further aspect, the disclosed compositions include one of the following cannabinoids encompassed by Formula (I):

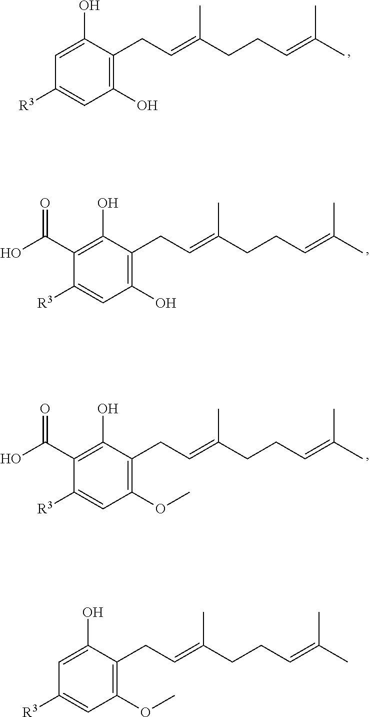

where R³ is C3-C5 alkyl; or a salt thereof. The C3-C5 alkyl can be any of the C3-C5 alkyl groups described above.

In specific aspects, the cannabinoid encompassed by Formula (I) is cannabigerovarin (CBGV), cannabigerovarinic acid (CBGVA), cannabigerol (CBG), cannabigerolic acid (CBVA), O-methylcannabigerol, cannabigerolic acid methylether, or any combination of these cannabinoids. The structure of these exemplary cannabinoids is shown below. For any of the cannabinoids below, and in general, any described cannabinoid, the carbon-carbon double bonds can be in the cis or trans configuration, but in some aspects, the carbon-carbon double bonds are present in the trans configuration as shown in the chemical structures.

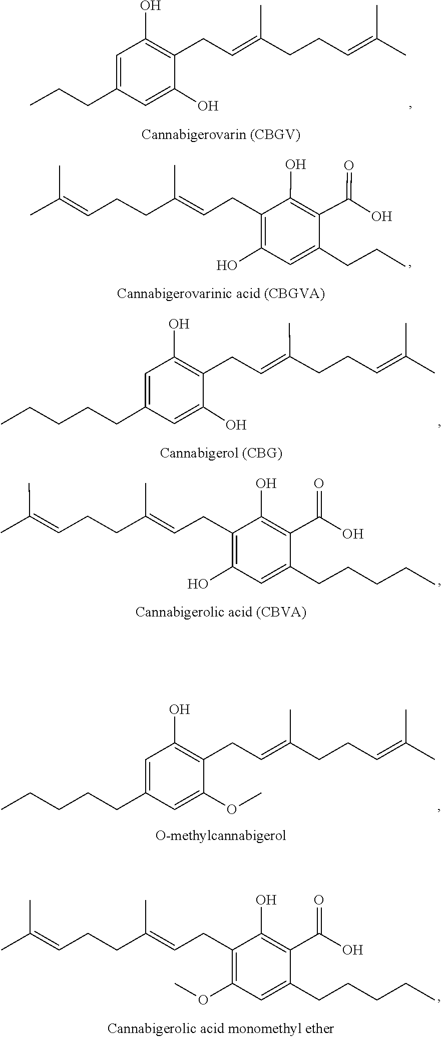

In one specific aspect, the cannabinoid encompassed by Formula (I) is cannabigerol (CBG):

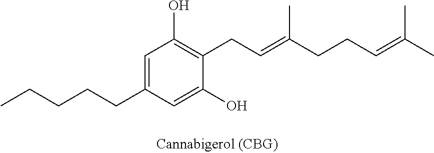 [SMILES: CCCCCc1cc(O)c(C/C=C(\C)CCC=C(C)C)c(O)c1]

In a further specific aspect, the cannabinoid encompassed by Formula (I) is cannabigerovarin (CBGV):

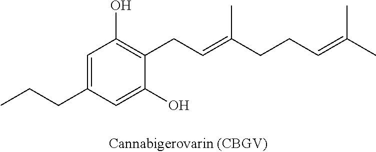 [SMILES: CCCc1cc(O)c(C/C=C(\C)CCC=C(C)C)c(O)c1]

In some aspects, the disclosed compositions include the cannabinoid of Formula (I), including any of the specific cannabinoids described above, together with a compound represented by Formula (II):

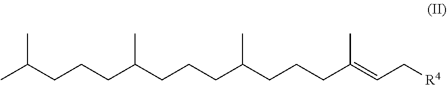

wherein R⁴ is a halide or —OR⁵; wherein R⁵, when present, is hydrogen or —COR⁶; wherein R⁶, when present, is C1-C18 alkyl, or aryl.

In a further aspect, the disclosed compositions include the cannabinoid of Formula (I), including any of the specific cannabinoids described above, combined with a compound represented by a compound within Formula (II) that has the formula:

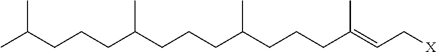

wherein X is a halide such as F, Cl, Br, or I.

In a further aspect, the disclosed compositions include the cannabinoid of Formula (I), including any of the specific cannabinoids described above, combined with a compound represented by a compound within Formula (II) that has the formula:

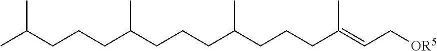

wherein R⁵, when present, is hydrogen or —COR⁶; wherein R⁶, when present, is C1-C18 alkyl, or aryl.

In a further aspect, the disclosed compositions include the cannabinoid of Formula (I), including any of the specific cannabinoids described above, combined with phytol, which has the following formula (and is encompassed within Formula (II):

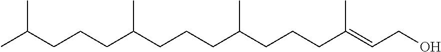 [SMILES: C/C(=C\CO)CCCC(C)CCCC(C)CCCC(C)C]

In a further aspect, the disclosed compositions include the cannabinoid of Formula (I), including any of the specific cannabinoids described above, combined with a compound represented by a formula (which is encompassed within Formula (II)):

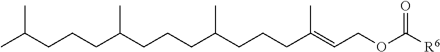

wherein R⁶, when present, is C1-C18 alkyl, or aryl. The C1-C18 alkyl can be methyl, ethyl, n-propyl, isopropyl, n-butyl, isobutyl, s-butyl, t-butyl, n-pentyl, isopentyl, s-pentyl, neopentyl, hexyl, heptyl, octyl, nonyl, decyl, dodecyl, tetradecyl, hexadecyl, eicosyl, tetracosyl, and others. The alkyl group can also be substituted or unsubstituted. In some aspects, the alkyl group can be substituted with one or more groups including amino, ether, halide, hydroxy, nitro, silyl, among others. The aryl group can be benzene, naphthalene, phenyl, biphenyl, or anthracene, among others. The aryl group can be substituted or unsubstituted. The aryl group can be substituted with one or more groups including alkyl, cycloalkyl, alkoxy, alkenyl, cycloalkenyl, alkynyl, cycloalkynyl, aryl, heteroaryl, or aldehy, among others.

In some aspects, the disclosed compositions include the cannabinoid of Formula (I), including any of the specific cannabinoids described above, combined with a terpene, with or without the compound of Formula (II). In various further aspects, the terpene is (E)-β-caryophyllene, (Z)-β-caryophyllene, caryophyllene oxide, α-humulene, myrcene, limonene, linalool, or pinene:

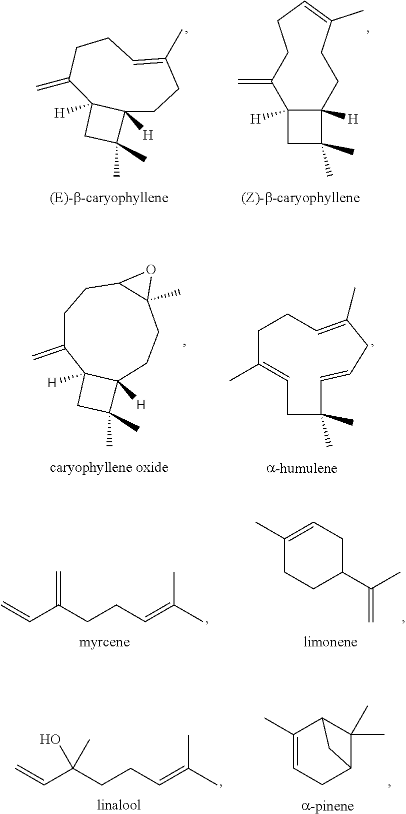

In a further aspect, the terpene in the composition is (E)-β-caryophyllene:

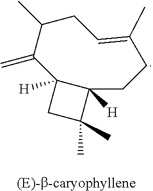

In a further aspect, the compound represented by Formula (II) or the terpene are present at a non-naturally occurring molar ratio. In a yet further aspect, the compound represented by Formula (II), or the terpene, are present at a molar amount that exceeds the molar amount of the cannabinoid. In general, although strains of Cannabis sativa differ in their substituents and quantities of these substituents, compounds within Formula (II), including phytol, are present in very small quantities in the plant (if present at all), typically resulting from the breakdown of plant matter. Similarly, some of the cannabinoids of Formula (I), if present at all in a strain of Cannabis sativa that has not been processed unnaturally (e.g., combusted), are present in amounts that are significantly different than the quantities described here with respect to the compositions and methods.

In a further aspect, the cannabinoid and the compound represented by Formula (II), or the terpene, are present at a molar ratio of about 1:5-1:10. Thus, in various aspects, the cannabinoid of Formula (I) and the compound of Formula (II), or the terpene, are present at a molar ratio of about 1:5, 1:6, 1:7, 1:8, 1:9, or 1:10. These specific molar ratios are different than any ratios occurring naturally, to the extent that the combination of compounds even occurs naturally at all.

In a further aspect, the cannabinoid of Formula (I) and the compound represented by Formula (II), or the terpene, are present at a molar ratio of about 1:5. In a specific aspect, the cannabinoid of Formula (I), for example CBG, and phytol (which is encompassed by Formula (II), or a terpene such as (E)-β-caryophyllene, are present in the composition at a molar ratio of about 1:5.

In various aspects, the compositions are substantially free of of Δ⁹-tetrahydrocannabinol (Δ⁹-THC), Δ⁸-tetrahydrocannabinol (Δ⁸-THC), Δ⁸-tetrahydrocannabiphorol (Δ⁸-THCP), Δ⁹-tetrahydrocannabiphorol (Δ⁹-THCP), cannabichromene (CBC), cannabicyclol (CBL), cannabidiol (CBD), cannabidiphorol (CBDP), cannabielsoin (CBE), cannabinidiol (CBND), cannabinol (CBN), and cannabitriol (CBT):

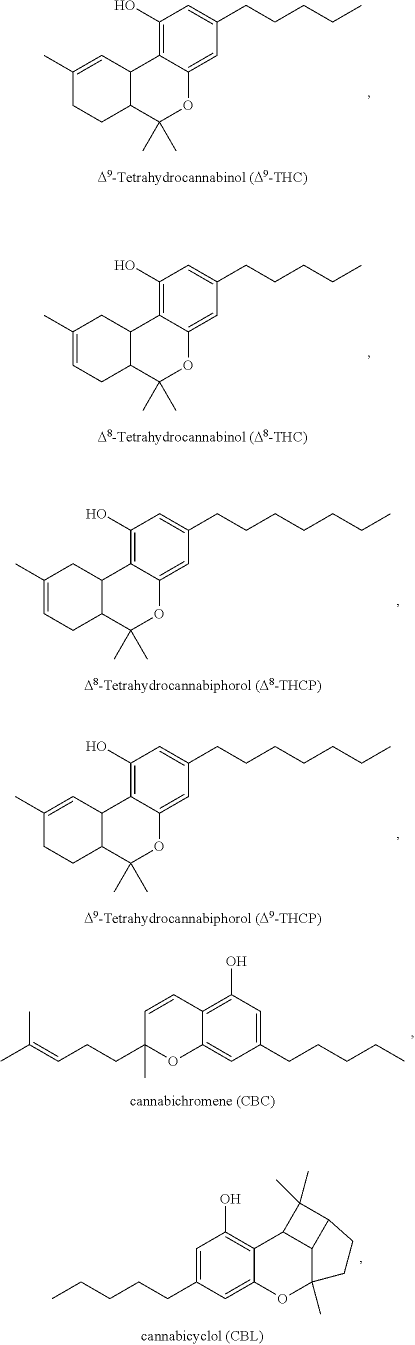

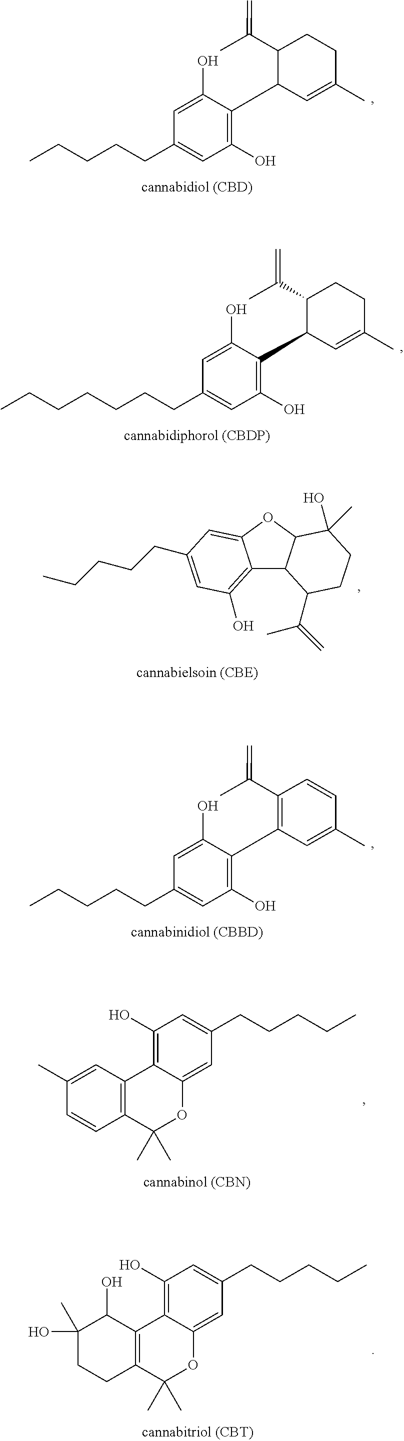

Thus, in various further aspects, the compositions contain less than about 10% by weight, less than about 5%, less than about 1%, less than about 0.5%, less than about 0.1%, less than about 0.05%, or less than about 0.01% by weight of Δ⁹-THC, Δ⁸-THC, Δ⁸-THCP, Δ⁹-THCP, CBC, CBL, CBD, CBDP, CBE, CBND, CBN, and CBT based on the total weight of the composition. In a further aspect, the composition is free of any measurable amount of Δ⁹-THC, Δ⁸-THC, Δ⁸-THCP, Δ⁹-THCP, CBC, CBL, CBD, CBDP, CBE, CBND, CBN, and CBT.

In various aspects, the compositions are substantially free of Δ⁹-tetrahydrocannabinol (Δ⁹-THC) and Δ⁸-tetrahydrocannabinol (Δ⁸-THC). Thus, in various further aspects, the compositions contain less than about 10% by weight, less than about 5%, less than about 1%, less than about 0.5%, less than about 0.1%, less than about 0.05%, or less than about 0.01% by weight of Δ⁹-THC and Δ⁸-THC based on the total weight of the composition. In a further aspect, the composition is free of any measurable amount of Δ⁹-THC and Δ⁸-THC.

In various aspects, the compositions are substantially free of Δ⁹-THC. Thus, in various further aspects, the compositions contain less than about 10% by weight, less than about 5%, less than about 1%, less than about 0.5%, less than about 0.1%, less than about 0.05%, or less than about 0.01% by weight of Δ⁹-THC based on the total weight of the composition. In a further aspect, the composition is free of any measurable amount of Δ⁹-THC.

In various aspects, the compositions are substantially free of Δ⁸-THC. Thus, in various further aspects, the compositions contain less than about 10% by weight, less than about 5%, less than about 1%, less than about 0.5%, less than about 0.1%, less than about 0.05%, or less than about 0.01% by weight of Δ⁸-THC based on the total weight of the composition. In a further aspect, the composition is free of any measurable amount of Δ⁸-THC.

In various aspects, the compositions are substantially free of any cannabinoid other than a cannabinoid encompassed by Formula (I). Thus, in various further aspects, the compositions contain less than about 10% by weight, less than about 5%, less than about 1%, less than about 0.5%, less than about 0.1%, less than about 0.05%, or less than about 0.01% by weight of any cannabinoid other than a cannabinoid encompassed by Formula (I) based on the total weight of the composition. In a further aspect, the composition is free of any measurable amount of any cannabinoid other than a cannabinoid within Formula (I). In another aspect, the cannabinoid in the composition consists essentially of a cannabinoid within Formula (I). In a further aspect, the cannabinoid in the composition consists of a cannabinoid within Formula (I).

In various aspects, the compositions consist essentially of the cannabinoid represented by Formula (I) and the compound represented by Formula (II), or the terpene. “Consists essentially of” in this context has its understood meaning, i.e., that the composition is limited to the recited components of the composition and any additional components that do not affect the basic and novel characteristics of the composition. This implies for example that compositions “consisting essentially of” the stated components are limited to those components and any other ingredients that do not materially affect any one of the following properties: (1) the ability of an effective amount of the composition to treat or prevent esophageal adenocarcinoma; (2) the ability to affect any desired therapeutic outcome (including delaying progression of or preventing a condition or disorder) in a subject that has or has been diagnosed with esophageal dysplasia, esophageal metaplasia, Barrett's Esophagus, gastroesophageal reflux disorder (GERD), or a condition associated with acid-biliary reflux; (3) the ability of an effective amount of the composition to counteract deoxycholic acid (DCA)-mediated mitochondrial or DNA damage in an esophageal cell (including in vivo and in vitro methods); (4) the ability of an effective amount of the composition to induce mitochondrial membrane depolarization in a cell, including esophageal cells such as squamous esophageal cells; (5) the ability of an effective amount of the composition to decrease DNA damage in a cell, including esophageal cells such as squamous esophageal cells; (6) the ability of an effective amount of the composition to reduce cell proliferation of a cell exposed to deoxycholic acid (DCA), including esophageal cells such as squamous esophageal cells; and (7) the ability of an effective amount of the composition to reduce or prevent low pH or bile-induced reactive oxygen species (ROS) in an esophageal cell, including squamous esophageal cells. As one of skill will appreciate, the basic and novel properties of the compositions are context specific, depending generally on the desired use.

In a further aspect, the composition consists of the cannabinoid of Formula (I) together with the compound of Formula (II), or the terpene, in some aspects at a non-naturally occurring molar ratio, e.g., 1:5.

In various aspects, the disclosed compositions comprise the cannabinoid represented by Formula (I), or the compound of Formula (II) or the terpene, present as an acceptable, non-naturally occurring salt. Thus, any naturally occurring cannabinoid represented by Formula (I), or the compound of Formula (II) or the terpene, present in the composition can be present as a non-naturally occurring acid or base salt. Illustrative examples of acceptable salts are mineral acid (hydrochloric acid, hydrobromic acid, phosphoric acid, and the like) salts, organic acid (acetic acid, propionic acid, glutamic acid, citric acid and the like) salts, and quaternary ammonium (methyl iodide, ethyl iodide, and the like) salts.

Acceptable salts can be prepared by reaction of the cannabinoid with a mineral or organic acid or an inorganic base, such as salts including sulfates, pyrosulfates, bisulfates, sulfites, bisulfites, phosphates, monohydrogenphosphates, dihydrogenphosphates, metaphosphates, pyrophosphates, chlorides, bromides, iodides, acetates, propionates, decanoates, caprylates, acrylates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, xylenesulfonates, phenylacetates, phenylpropionates, phenylbutyrates, citrates, lactates, β-hydroxybutyrates, glycolates, tartrates, methane-sulfonates, propanesulfonates, naphthalene-1-sulfonates, naphthalene-2-sulfonates, and mandelates.

According to one aspect, if the cannabinoid has one or more acidic functional groups, the desired salt can be prepared by any suitable method known in the art, for example, treatment of the free acid with an inorganic or organic base, such as an amine (primary, secondary or tertiary), an alkali metal hydroxide or alkaline earth metal hydroxide, or the like. It is understood that the acceptable salts are non-toxic and suitable for ingestion. Additional information on suitable acceptable salts can be found in Remington's Pharmaceutical Sciences, 17th ed., Mack Publishing Company, Easton, Pa., 1985, which is incorporated by reference.

In various aspects, a product prepared from a disclosed composition can comprise a cannabinoid represented by Formula (I) and a compound represented by Formula (II), or the terpene, present together with an acceptable, non-naturally occurring carrier. Various suitable non-naturally occurring carriers are described in Remington's Pharmaceutical Sciences, 17th ed., Mack Publishing Company, Easton, Pa., 1985. Non-limiting examples include non-naturally occurring polymeric carriers or binders in liquid or solid form, such as polyglycolic acids, synthetic polymers, non-naturally occurring conjugates of proteins, and the like.

In various aspects, the disclosed compositions are formulated as an oral dosage form. In preparing the compositions for oral dosage form, any convenient pharmaceutical media can be employed. For example, water, glycols, oils, alcohols, flavoring agents, preservatives, coloring agents and the like can be used to form oral liquid preparations such as suspensions, gels, elixirs, and solutions; while carriers such as starches, sugars, microcrystalline cellulose, diluents, granulating agents, lubricants, binders, disintegrating agents, and the like can be used to form oral solid preparations such as powders, capsules, and tablets. In one aspect, the oral dosage form is formulated such that the composition can be ingested and effectively adhere or coat a portion of the esophagus, to ensure adequate absorption of the oral dose into relevant esophageal cells.

### C. METHODS OF USING THE CANNABINOID COMPOSITIONS

The disclosed compositions are useful in treating or preventing esophageal adenocarcinoma in a subject. The disclosed compositions are also useful in treating or preventing diseases such as esophageal dysplasia, esophageal metaplasia, Barrett's Esophagus, gastroesophageal reflux disorder (GERD), or other conditions associated with acid-biliary reflux.

Thus, in one aspect, disclosed are methods for treating or preventing esophageal adenocarcinoma in a subject, the methods comprising administering to the subject a therapeutically effective amount of a composition comprising: (a) a cannabinoid represented by Formula (I); and (b) a compound represented by Formula (II), or a terpene. In a further aspect, the subject has or has been diagnosed with esophageal dysplasia, esophageal metaplasia, Barrett's Esophagus, gastroesophageal reflux disorder (GERD), or a condition associated with acid-biliary reflux.

In a further aspect, disclosed are methods for treating or preventing esophageal dysplasia, esophageal metaplasia, Barrett's Esophagus, gastroesophageal reflux disorder (GERD), or a condition associated with acid-biliary reflux in a subject, the methods comprising administering to the subject a therapeutically effective amount of a composition comprising: (a) a cannabinoid represented by Formula (I); and (b) a compound represented by Formula (II), or a terpene.

In a further aspect, the compositions can be administered to the subject via oral administration (e.g., as a tablet, capsule, lozenge, or troche). Administration can be continuous or intermittent. A preparation can be administered therapeutically; that is, administered to treat or ameliorate an existing disorder or condition.

The effective amount or dosage of the composition can vary within wide limits. Such a dosage is adjusted to the individual requirements in each particular case including the specific composition(s) being administered and the condition being treated, as well as the patient being treated. In general, single dose compositions can contain such amounts or submultiples thereof of the composition to make up the daily dose. The dosage can be adjusted by the individual physician in the event of any contraindications. Dosage can vary, and can be administered in one or more dose administrations daily, for one or several days. In some aspects, the effective amount is a therapeutically effective amount. In a further aspect, the effective amount is a prophylactically effective amount.

In a further aspect, the subject is a mammal. In a still further aspect, the mammal is a human. In a still further aspect, the subject has been diagnosed with a need for treatment of esophageal adenocarcinoma or another relevant condition prior to the administering step. In a further aspect, the subject is at risk for developing esophageal adenocarcinoma or another relevant condition prior to the administering step. In a further aspect, the method further comprises the step of identifying a subject in need of treatment of esophageal adenocarcinoma or another relevant condition. In a still further aspect, the subject has been diagnosed with a need for treatment of esophageal dysplasia, esophageal metaplasia, Barrett's Esophagus, gastroesophageal reflux disorder (GERD), or a condition associated with acid-biliary reflux prior to the administering step. In a further aspect, the subject is at risk for developing esophageal adenocarcinoma prior to the administering step. In a further aspect, the method further comprises the step of identifying a subject in need of treatment of esophageal dysplasia, esophageal metaplasia, Barrett's Esophagus, gastroesophageal reflux disorder (GERD), or a condition associated with acid-biliary reflux.

In addition to in vivo methods, the compositions can be used in vitro (in vivo) to target particular cellular pathologies. In one aspect, disclosed is a method of counteracting deoxycholic acid (DCA)-mediated mitochondrial or DNA damage in an esophageal cell, the method comprising contacting the esophageal cell with an effective amount of the composition of a cannabinoid represented by Formula (I) and a compound represented by Formula (II), or a terpene.

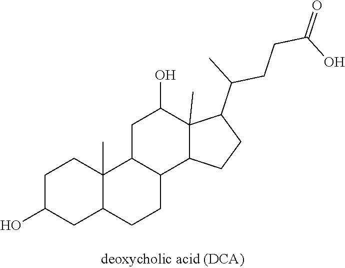 [SMILES: CC(CCC(=O)O)C1CCC2C3CCC4CC(O)CCC4(C)C3CC(O)C12C]

In a further aspect, disclosed is a method of counteracting deoxycholic acid (DCA)-mediated mitochondrial or DNA damage in an esophageal cell, the method comprising contacting the esophageal cell with an effective amount of the composition of cannabigerol (CBG) and phytol (Phy). In a further aspect, the cell is mammalian. In a still further aspect, the cell is a human cell. In a still further aspect, the cell is an esophageal cell. In yet a further aspect, the cell has been isolated from a human prior to the contacting step. In a further aspect, contacting the cell is via administration to a subject.

In one aspect, disclosed is a method of inducing mitochondrial membrane polarization in a cell, the method comprising contacting the cell with an effective amount of the composition of a cannabinoid represented by Formula (I) and a compound represented by Formula (II), or a terpene. In a further aspect, disclosed is a method of inducing mitochondrial membrane polarization in a cell, the method comprising contacting the cell with an effective amount of the composition of cannabigerol (CBG) and phytol (Phy). In a further aspect, the cell is mammalian. In a still further aspect, the cell is human. In a still further aspect, the cell is an esophageal epithelial cell. In yet a further aspect, the cell has been isolated from a human prior to the contacting step.

In one aspect, disclosed is a method of decreasing DNA damage in a cell, the method comprising contacting the cell with an effective amount of the composition of a cannabinoid represented by Formula (I) and a compound represented by Formula (II), or a terpene. In a further aspect, disclosed is a method of decreasing DNA damage in a cell, the method comprising contacting the cell with an effective amount of the composition of cannabigerol (CBG) and phytol (Phy). In a further aspect, the cell is mammalian. In a still further aspect, the cell is human. In a still further aspect, the cell is an esophageal epithelial cell. In yet a further aspect, the cell has been isolated from a human prior to the contacting step.

In one aspect, disclosed is a method of reducing cell proliferation in a cell exposed to deoxycholic acid (DCA), the method comprising contacting the cell with an effective amount of the composition of a cannabinoid represented by Formula (I) and a compound represented by Formula (II), or a terpene. In a further aspect, disclosed is a method of reducing cell proliferation in a cell exposed to deoxycholic acid (DCA), the method comprising contacting the cell with an effective amount of the composition of cannabigerol (CBG) and phytol (Phy). In a further aspect, the cell is mammalian. In a still further aspect, the cell is human. In a still further aspect, the cell is an esophageal epithelial cell. In yet a further aspect, the cell has been isolated from a human prior to the contacting step.

In one aspect, disclosed is a method of reducing or preventing low pH or bile-induced reactive oxygen species (ROS) in a cell, the method comprising contacting the cell with an effective amount of the composition of a cannabinoid represented by Formula (I) and a compound represented by Formula (II), or a terpene. In a further aspect, disclosed is a method of reducing or preventing low pH or bile-induced reactive oxygen species (ROS) in a cell, the method comprising contacting the cell with an effective amount of the composition of cannabigerol (CBG) and β-caryophyllene. In a further aspect, the cell is mammalian. In a still further aspect, the cell is human. In a still further aspect, the cell is an esophageal cell. In yet a further aspect, the cell has been isolated from a human prior to the contacting step.

### D. EXAMPLES

The following examples are put forth so as to provide those of ordinary skill in the art with a complete disclosure and description of how the methods and products claimed are made and evaluated, and are intended to be purely exemplary and are not intended to limit the scope of the claims. Efforts have been made to ensure accuracy with respect to numbers (e.g., amounts, temperature, etc.) but some errors and deviations should be accounted for. Unless indicated otherwise, parts are parts by weight, temperature is in ° C. or is at ambient temperature, and pressure is at or near atmospheric. The Examples should not be construed as limiting.

### 1. Methods and Materials

The cannabinoids referenced in the following examples were purchased from Cayman Chemical and brought up to a stock concentration of 100 mM in DMSO. Terpenes were purchased from Sigma Aldrich. Bile acid cocktail consisted of an equimolar mixture of glycocholate, taurocholate, glycodeoxycholate, glycochenodeoxycholate, and deoxycholate at a final concentration of 0.2 mM. This cocktail reflects the mixture of bile acids to which the distal esophagus is ordinarily exposed during gastroesophageal reflux

The following examples utilized three human primary esophageal epithelial: HET1a (ATCC, cat #CRL-2692, Virginia, USA), Human Esophageal Epithelial Cells (Sciencell, cat #2720, California, USA), and Human Primary Esophageal Epithelial Cells (CellBiologics, cat #H-6046, Illinois, USA). Flol cells were also used in experiments as a human esophageal adenocarcinoma cancer cell model. Frozen aliquots were plated in T-25 flasks pre-coated with 0.01 mg/mL human fibronectin (Corning, cat #356008, New York, USA) and 0.03 mg/mL Collagen I, bovine (ChemCruz, cat #sc-29009, Texas, USA) with Bronchial Epithelial Cell Growth Medium (Lonza, Cat #CC-3170, Basel, Switzerland) at 37° C. in a CO₂ incubator. When cells reached 70% confluency, they were enzymatically disaggregated with TrypLE (Life Technologies, Cat #12604-013, California, USA) for 10 minutes at 37° C. The cells were spun down (300 times g for 5 minutes) and plated in T-75 flasks pre-coated with fibronectin and collagen I to expand the number of cells. In the following examples, cells were plated in 6-well dishes pre-coated with fibronectin and collagen I. CP-A Barrett's esophagus cells (ATCC) were maintained in MCDB-153 supplemented with 0.4 μg/ml hydrocortisone, 20 ng/ml recombinant human epidermal growth factor, 8.4 μg/L cholera toxin, 20 mg/L adenine, 140 μg/ml bovine pituitary extract, lx ITS Supplement (Sigma; I1884), 4 mM glutamine and 5% fetal bovine serum.

Cells were cultured prior to exposure to the indicated test articles. After treatment, as indicated, cells were washed and resuspended in a phenol red-free RPMI or DMEM and subsequently treated with the XTT assay (ThermoFisher, Waltham, MA, USA) or the MTS assay (Promega, Madison, WI, USA) following manufacturer protocols.

To assess the expression of CB1, the following examples utilized a tissue array containing 50 cases/50 cores of esophageal adenocarcinoma, cardia adenocarcinoma, and normal esophageal and cardia tissue was obtained from Biomax (cat #, BC001113, Rockville, MD, USA). The tissue sections were then rehydrated through a series of ethanol solutions and placed in distilled water. Antigen retrieval was performed in sodium citrate pH 6.0 (Sigma, S-4641, St. Louis, MO, USA) using an electric pressure cooker. The tissue sections were placed in a solution of 0.1% TritonX-100 (Sigma, T9284) in phosphate buffered saline (PBS) for 15 minutes and pre-blocked with hydrogen peroxide Blocking Reagent (Abcam, 64218, Cam-bridge, UK). Blocking solution consisting of 10% normal donkey serum in PBS (EMD Millipore, S30-100 ML, Billerica, MA, USA) was added to the slides and left for 30 minutes at room temperature. The blocking solution was replaced with the primary antibodies diluted in blocking solution using rabbit anti-CB1 (clone D5N5C, Cell Signaling Technologies, cat #93815, Massachusetts, USA) and anti-epithelial cell adhesion molecule (EPCAM) (Origene, cat #UM500096, Maryland, USA), which was used to mark EAC. The tissue sections were incubated in primary antibody solution overnight at 4° C., and washed twice with 0.1% Tween-20 (Promega, H5151, Madison, WI, USA) in PBS. Sections were then incubated with secondary antibody (donkey anti-rabbit IgG-594 and donkey anti-mouse IgG-488) diluted 1:500 in PBS for 1 hour at RT and washed twice in 0.1% Tween-20 in PBS for 15 minutes each. 4′,6-diamidino-2-phenylindole (DAPI) was used to stain nuclei. Semi-quantitative analysis was performed using an H-score analysis by two independent observers. The proportion (0-100) and intensity of CB1 immunostaining (0: no staining; 1: weak staining; 2: moderate staining, 3: strong staining) were used to calculate an H-score.

To determine the cellular location of CB1, cells were cultured overnight in Nunc Lab-Tek II Chamber Slides (ThermoFisher, cat #154461) pre-coated with fibronectin and collagen I. The next day, cells were briefly fixed with 1:1, 4% paraformaldehyde: media for 2 minutes followed by 4% paraformaldehyde for 15 minutes. Cells were washed twice with PBS for 15 minutes and permeabilized with Triton X-100 (Sigma-Aldrich, cat #T8787, Missouri, USA) in PBS for 30 minutes. Cells were blocked with 5% donkey serum in PBS for 30 minutes. Primary antibodies that recognize CB1 (Bioss Antibodies, cat #bs-1683R-A488, Massachusetts, USA) and CB1 Receptor (D5N5C) (Cell Signaling Technologies, cat #93815, Massachusetts, USA). MitoTracker Red CMXRos (ThermoFisher, cat #M7512, Massachusetts, USA) and 4′, 6-diamidino-2-phenylindole (DAPI) was used to stain nuclei. Images were acquired using a Leica True Confocal Scanning System SP8 equipped with GaAsP HyD detectors and an HC Plan APO 63×/1.40 NA, oil objective.

RNA was isolated from cultured cells using the RNAeasy mini kit (Qiagen, Maryland, USA), then converted to cDNA using Superscript III cDNA kit (Life Technologies, California). qPCR was performed using qSTART qPCR primers against Homo sapiens genes GAPDH (cat #HP205798) and CNR1/CB1 (cat #HP227608). All qPCR reactions were done using the standard SYBR green protocol of QuantStudio 6 Flex Real-Time PCR System (ThermoFisher, Massachusetts, USA).

MitoProbe JC-1 Assay Kit for Flow Cytometry (cat #M34152, ThermoFisher, Massachusetts, USA) was used to measure the mitochondrial membrane potential. Depending on the experimental group, cells were pre-treated with the CBG/Phytol admixture or DMSO and incubated for 2 hours at 37° C. in a CO2 incubator. Cells were then treated with DCA with the CBG/Phytol admixture or DMSO. After treatment, cell were loaded with 2 μM of JC-1 and incubated for 15 minutes at 37° C. Cytoplasmic JC-1 monomers were detected in the green spectrum (˜529 nm) while mitochondrial J-aggregates were detected in the red spectrum (˜590 nm). Results are representative of 2 and 3 independent repeats per cell line.

ROS production by mitochondria was measured by fluorescence microscopy using the MitoSOX Red reagent (ThermoFisher, cat #M36008, Massachusetts, USA). Depending on the experimental group, cells were pre-treated with the CBG/Phytol admixture or DMSO and incubated for 2 hours at 37° C. in a CO₂ incubator. After, cells were incubated in a 5 μM solution of MitoSOX Red reagent prepared in Hank's Balance Salt Solution (ThermoFisher, cat #24020117, Massachusetts, USA) and incubated at 37° C. for 10 minutes. Cells were washed with warmed HBSS and imaged using a Nikon C2 Confocal System for Eclipse TiE microscope with perfect focus using Plan APO 20 times/0.75 NA and 60 times/1.40 NA objectives. DCA was added to the media after 2 minutes of incubation and the 580 nm fluorescence signal was capture every second for 20-25 minutes. NIS elements analysis package was used to measure the signal intensity and excel was used to calculate the rate change.

The activation of ATM and H2AX (a marker of DNA breaks) was measured using the Muse Multi-Color DNA Damage kit (MilliporeSigma, Burlington, MA, USA) according to the manufacturer's instructions. The percentage of ATM activated cells and H2AX activated cells was determined as dual activation by monitoring expression of both the ATM and γ-H2AX, using the Muse Cell Analyzer (MilliporeSigma, Burlington, MA, USA). Expression of γ-H2AX was determined by flow cytometry on an Accuri C6 flow cytometer following manufacturer protocol (MilliporeSigma, Burlington, MA, USA).

The impact of phytocannabinoids on acquisition of new genomic changes over time in human esophageal cells was assessed using Axiom™ Precision Medicine Diversity Arrays (PMDA; Affymetrix). HET1a cells, plated in T-150 flasks at a seeding density of 2,500 cells per cm², were divided into 4 experimental groups (n=2 each): (i) DMSO-treated controls, (ii) treated with 100 μM of DCA alone, (iii) treated with CBG/Phytol admixture alone, and (iv) treated with 100 μM of DCA and CBG/Phytol admixture. DCA and/or CBG/Phytol were freshly prepared daily by dissolving in DMSO and then added to the media. These cells were cultured for 14 consecutive days. An aliquot of parental cells was collected and saved at the beginning of experiment to be used as “Day 0” or baseline genome. The DNA from cultured and “Day 0” cells was extracted using a QIAGEN DNeasy Blood & Tissue Kit (Qiagen, cat #69504, Maryland, USA) and hybridized to PMDA arrays. Genomic changes acquired by control and treated HET1a cells during their growth in culture were identified, using the genome of “Day 0” cells as baseline.

### 2. Example 1: Composition of CBG and Phytol Counteracts DCA-Mediated Mitochondrial and DNA Damage in Squamous Esophageal Cells

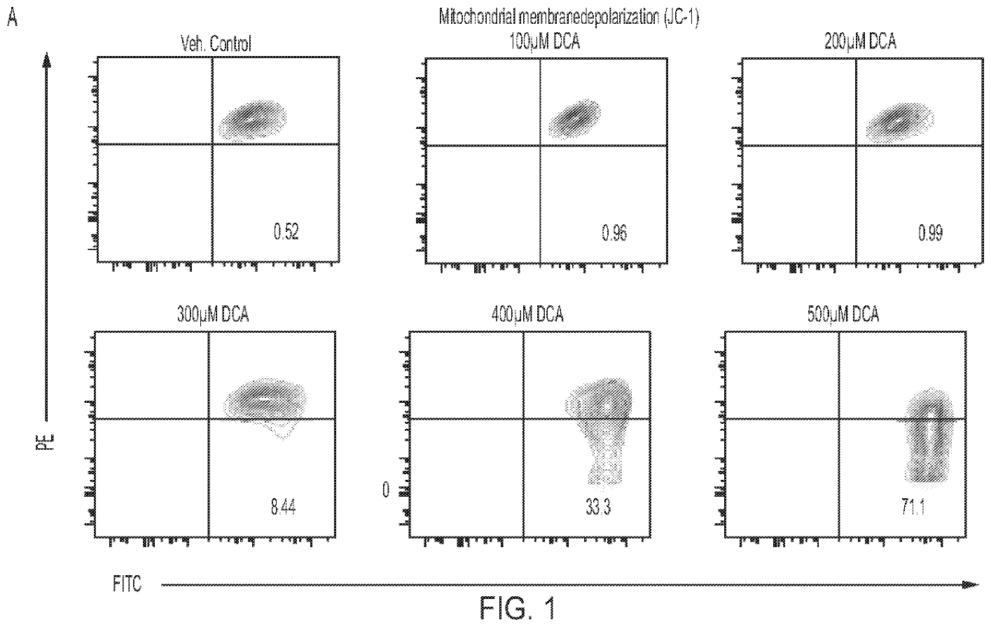

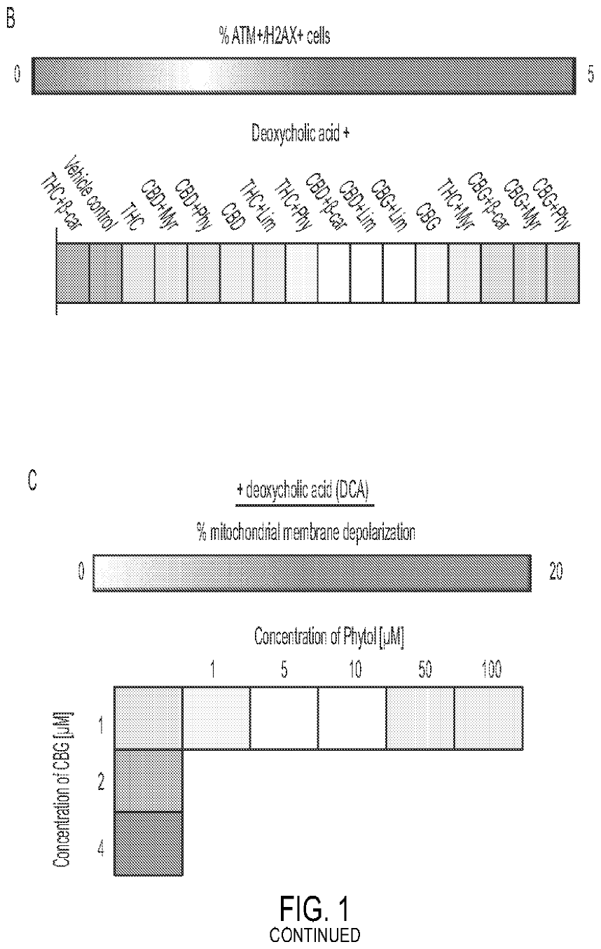

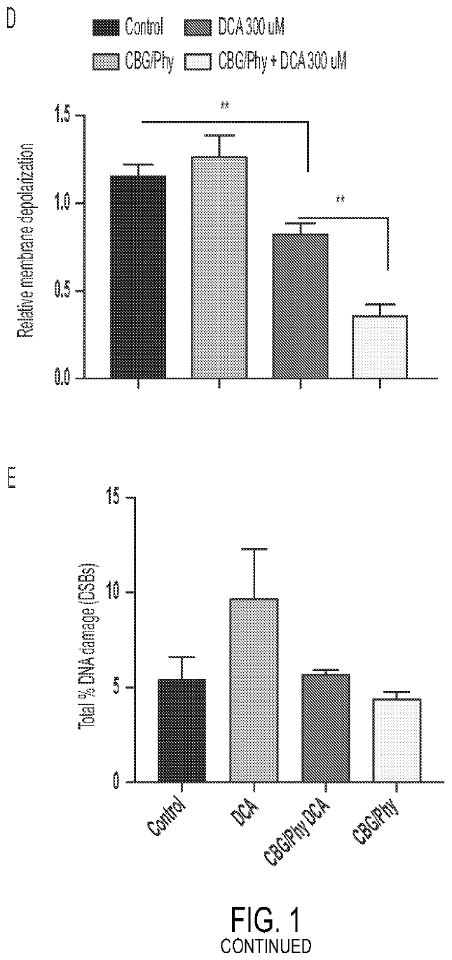

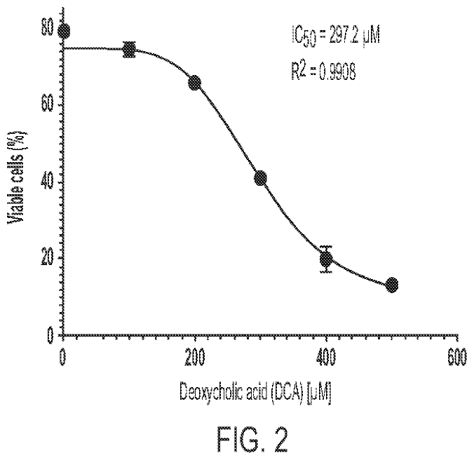

The lethal dose 50% (EC50) of DCA was established on an immortalized squamous epithelial cell line, HET1a. FIG. 2 shows cell viability, which was evaluated by XTT assay and is expressed as a % of vehicle control cohort, of HET1a following acute exposure to DCA at indicated doses. FIG. 1A shows the mitochondrial membrane polarization of HET1a as measured by red/green fluorescent changes (JC-1) after acute exposure to DCA in the concentration range of 100-500 mM. These concentrations of DCA are found in esophageal aspirates of patients with GERD. Doses as low as 100 μM DCA were found to be sufficient to induce membrane polarization. In both studies, dosing above 300 μM induced severe mitochondrial membrane depolarization and cellular apoptosis. Therefore, to study the toxicity-mitigating effect of the compounds, 100 μM DCA was determined to be an appropriate dose that would increase membrane depolarization and DNA damage without leading to cellular apoptosis.

Combinations of terpenes and cannabinoids were introduced to assess the effect on DNA damage using the Muse Multicolor DNA Damage Kit (see methods for details). Based on a preliminary screen shown in FIG. 1B, it was concluded that DCA induced the highest proportion of DNA damage, along with the addition of tetrahydrocannabinoid (THC) containing groups, >5% vs. an untreated group. Notably, the combination of cannabigerol (CBG), which comprises less than 1% of the total plant chemical and the terpenoid phytol (as well as myrcene or β-caryophyllene) optimally counterattacked the DCA-induced DNA damage indicated by the % ATM and H2AX positive cells.

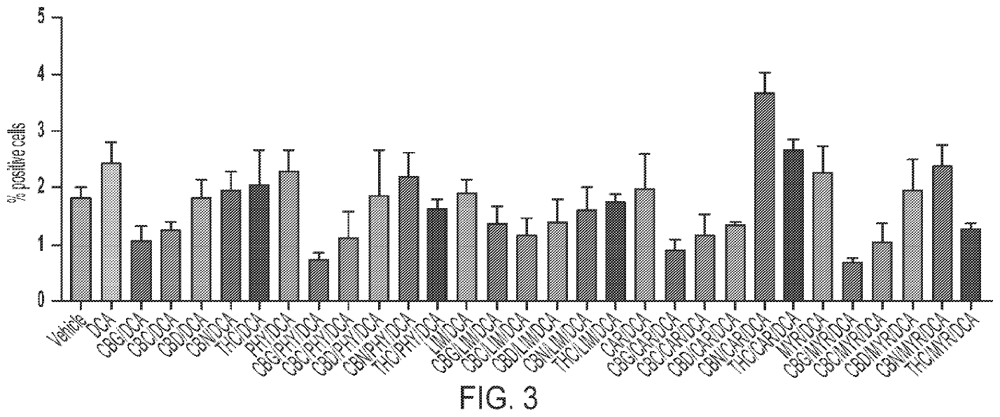

Various ratios of the combination of CBG and phytol were explored to assess DCA-induced mitochondrial membrane depolarization. As demonstrated in FIG. 1C-1D, the combination of CBG and phytol at a micromolar equivalency of 1:5 was determined to reduce the effect of DCA on membrane depolarization. This effect was not reproduced by either compound alone in a statistically significant manner (p<0.01, FIG. 1D). FIG. 1E shows a reduction of double strand breaks induced by DCA when the combination ratio (1:5) of CBG and phytol is employed. These findings were validated in the non-immortalized squamous epithelial cell line HEsEpiC, identifying CBG and phytol resulting in the lowest % positive cells with DCA-induced DNA damage. The results are shown in FIG. 3.

### 3. Example 2: Composition of CBG and Phytol Restricts Markers of Apoptosis Resistance in DCA-Treated Esophageal Cells

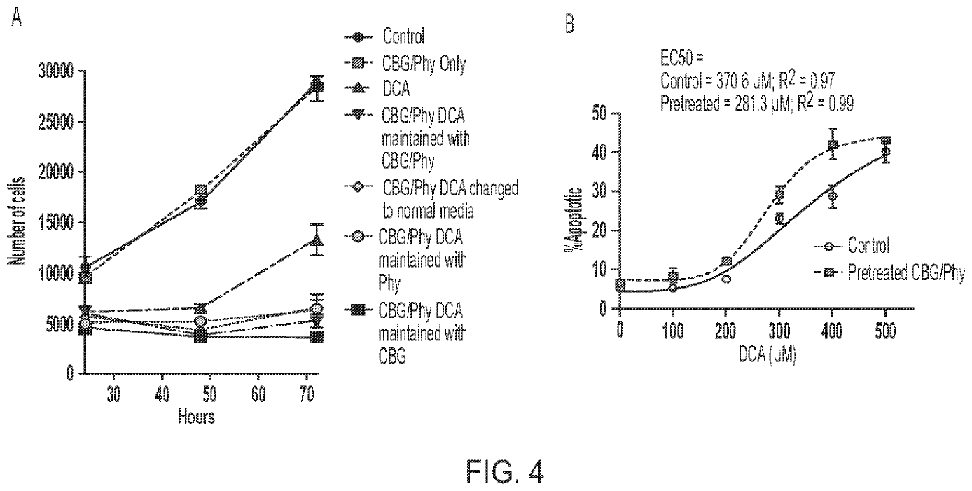

In this example, the role of CBG and Phytol on hallmarks of apoptosis resistance is described. FIG. 4A shows cell viability of HET1a cells treated with or without 100 mM DCA for 24 hours and with or without CBG and/or Phytol for 72 hours. Cells were pretreated with or without CBG/Phytol and then treated with DCA or a vehicle control for 24 hours. On the following day, the cells were incubated with fresh media, without DCA, and with or without CBG and/or Phytol. Cell viability was determined via XTT assay. It was confirmed that CBG/Phytol alone did not affect cell proliferation. DCA alone lead to significant reduction in number of cells by 48 hours, but the number of cells increased by 72 hours as observed on 72-hour time point. These results indicate that cells treated with DCA showed reduced cell proliferation or increased cellular apoptosis but were able to resume cell division after DCA was removed from the media. In contrast, cells pre-treated with CBG/Phytol admixture and exposed to DCA show reduced number of cells upwards of 72-hours. These findings indicate that the admixture significantly affects the ability of cells to recover from the cytotoxic effect of DCA.

Next, HET1A cells were treated with DCA ranging from 0-500 μM with or without pre-treating with the CBG/Phytol admixture. FIG. 4B is a plot representing the percentage of apoptotic cells following these treatments. The FITC Annexin Apoptosis Detection Kit was used to evaluate changes in apoptosis. These results show that pre-treatment with the admixture caused cells to become more susceptible to DCA-mediated apoptosis. Taken together, these two evidences suggest that apoptotic resistances and unrestricted proliferation following toxic insult are blunted by the cannabinoid-terpene combination.

### 4. Example 3: Composition of CBG and Phytol Counteracts DCA-Mediated Genomic Instability

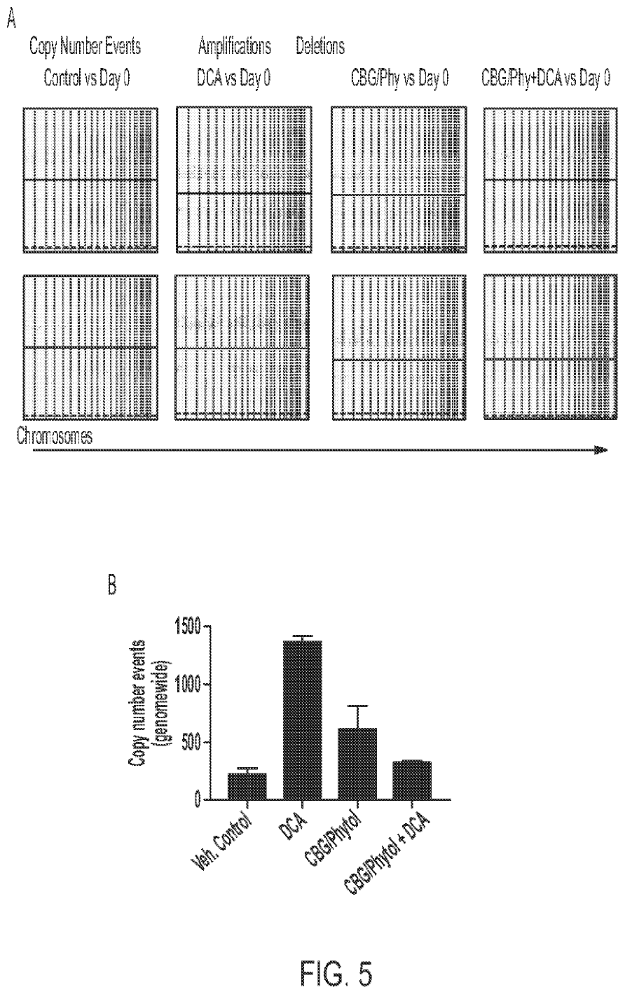

Based on the examples above, it was hypothesized that increased stress-induced apoptosis along with reduced DNA damage and mitochondrial disruption may be concordant to reduced genomic instability. To test this, HET1A cells were divided into 4 experimental groups: (i) vehicle-treated controls, (ii) 100 μM DCA alone, (iii) CBG/Phytol admixture alone (1:5), and (iv) 100 μM DCA and CBG/Phytol admixture (1:5). Cohorts were subjected to daily treatments for 14 days. An aliquot of parental HET1A cells was saved in the beginning of experiment to be used as Day 0 (baseline) genome. DNA from cultured and Day 0 cells was extracted and analyzed using PMDA arrays (Affymetrix). Genomic instability in cultured cells was assessed by identifying new copy number events (both deletions and amplifications), using genome of “Day 0” cells as baseline. Images showing the copy number events acquired by control and treated cells in three weeks, relative to day 0 cells, are found in FIG. 5A. FIG. 5B is a bar graph showing the total number of copy-number change events throughout the genome. Compared to control cells, DCA induced a marked (>6-fold) increase in genomic instability, as assessed from the acquisition of new copy number events over a period of three weeks. Although treatment with a mixture of CBG and Phytol also led to a moderate (>2-fold) increase in genomic instability, the cells treated with DCA in the presence of CBG and Phytol showed reduced genomic instability relative to control cells. These data demonstrate that treatment with CBG/Phytol reduces DCA-induced genomic instability. Taken together with the evidence from oxidative damage and induction of apoptosis, daily administration of CBG/Phytol admixture may counteract DCA-mediated DNA damage and genomic instability, a precursor to mutagenicity and cancer.

### 5. Example 4: Composition of CBG and β-Caryophyllene Mitigates Low pH and Bile Acid-Induced ROS in Barrett's Esophagus Cells

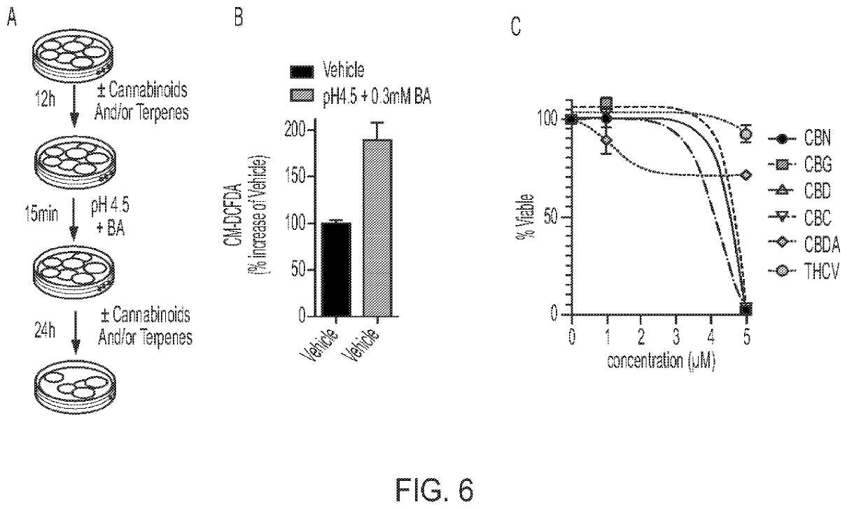

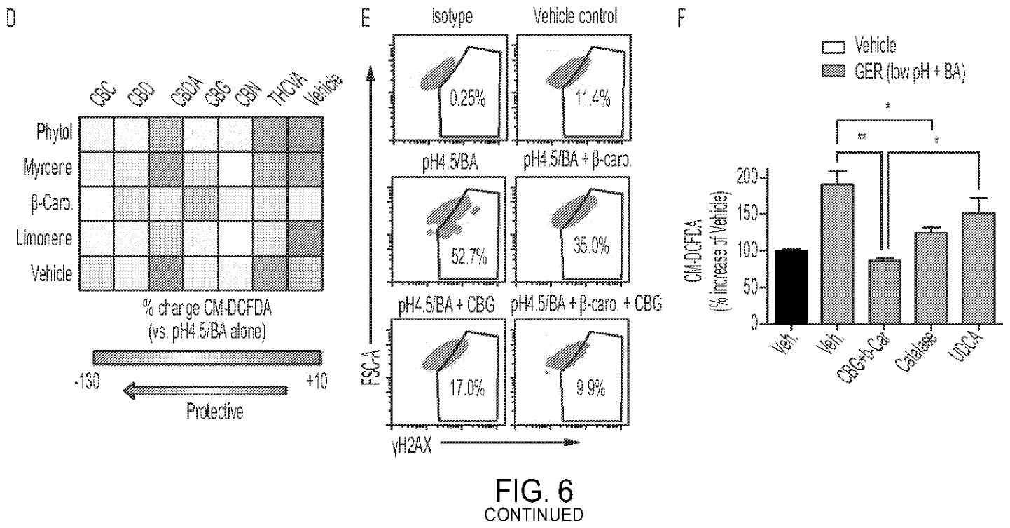

This example demonstrates the effect of cannabinoids and terpenes in metaplastic esophageal cells, which are considered a pre-malignant phenotype in the upper GI. Combinations of cannabinoids/terpenes were tested to determine whether they protected cells in the esophagus following acute exposure to a low pH environment (pH4.5) combined with a bile acid cocktail comprised of multiple physiologically-relevant secondary bile acids, which mimics the caustic environment during gastroesophageal reflux (GER). Following the experimental design in FIG. 6A, non-dysplastic BE cells (CP-A) were treated with a vehicle control or cannabinoids in the presence or absence of terpenes for 12 hours followed by an acute (15 minutes) exposure to low pH and bile acid cocktail. The cells were either immediately analyzed for ROS via fluorescent detection of CM-DCFDA or recovered for 24 hours and analyzed for DNA damage, via γ-H2AX. FIG. 6B shows the ROS results expressed as a % increase vs vehicle control. Acute exposure to the low pH/bile acid combination results in significant increase in ROS. Additionally, as shown in FIG. 6C, it was confirmed that 1μM cannabinoid did not confer an increase in cell death or effect on proliferation. Next, flow cytometry and a screen of various combinations of terpenes and cannabinoids at the 1:5 ratio were used to determine that while CBG and phytol reduced the amount of ROS caused by the low pH/bile acid insult, the highest reduction in ROS was identified in the combination of CBG and β-caryophyllene. FIG. 6D presents a heat map showing a matrix of ROS induced by GER in CP-A cells that were pre-treated with the indicated conditions and cannabinoid terpene combinations (1:5). Furthermore, the combination of CBG and β-caryophyllene was tested in the residual cells and harvested 24 h post-treatment for assessment of DNA damage to obtain the γ-H2AX fluorescence plots in FIG. 6E. A significant diminishment of DNA damage was observed. Lastly, other combinations that are known to reduce the toxic assault of GER were compared, including catalase and the tertiary bile acid ursodeoxycholic acid. As shown in FIG. 6F, the results suggested that CBG/β-car (1:5) mitigated the oxidative insult of GER to a degree significantly less than the protective effect of UDCA.

### 6. Example 5: Multiple G-Protein Coupled Receptors (GPCR) and Cannabinoid Receptor (CB-1) Affiliate with BE and EAC

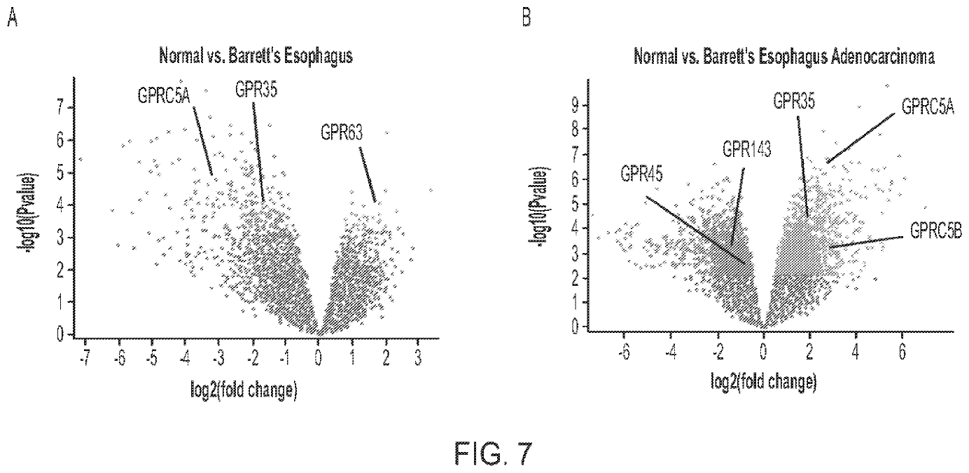

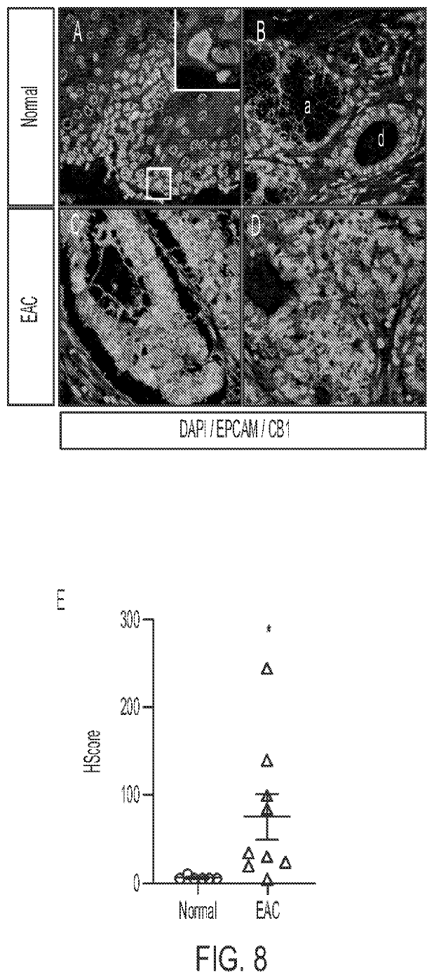

It is increasingly clear that the ECS functions through multiple receptor pathways that are CB-1 and CB-2 independent. Indeed, there is very little evidence to pinpoint the other G-protein couple receptors (GPCRs) that modulate endo- and phyto-cannabinoid activity and the resulting effect on the ECS. In an effort to provide translational relevance, the gene expression and protein profile of established or putative ECS receptors comparing normal, BE and/or adenocarcinoma lesions from human were examined. Indeed, GPCR including GPR3532 and GPR6333 and other putative GPCR orphan receptors are thought to retain affinity for and activity in response to endo- and phyto-cannabinoids. Firstly, the public data repository was queried for GSE142034, comparing normal squamous epithelium, BE metaplasia and adenocarcinoma (N=8 each). Over-expressed or under-expressed genes vs. normal epithelium (log 2FC> or <0.9) were evaluated statistically, and a cohort of GPCR that affiliated with the different disease types was identified. This analysis is shown in FIG. 7A-B. While GPR63 was found upregulated only in BE vs. normal, GPR35 and GPR5CA were found differentially up or down regulated in BE and EAC vs. normal. Finally, to study CB-1, tissues were stained with an anti-CB-1 antibody and anti-EPCAM antibody, a transmembrane glycoprotein expressed in various epithelial carcinomas including EAC. In the adjacent normal stratified squamous epithelium, punctate CB1-expression was observed mainly in the basal layer of the stratified squamous epithelium using confocal microscopy as shown in FIG. 8A-B. In comparison to normal tissue, increased expression of CB1 was observed in EPCAM-positive EAC tissue (FIG. 8C-D). Therefore, H-Score was used to perform a semiquantitative intensity scoring comparative analysis of CB1 expression between normal stratified squamous epithelial cells and EAC in FIG. 8E.

### 7. Example 6: Formulation of CBG-Phytol-Hydrogel

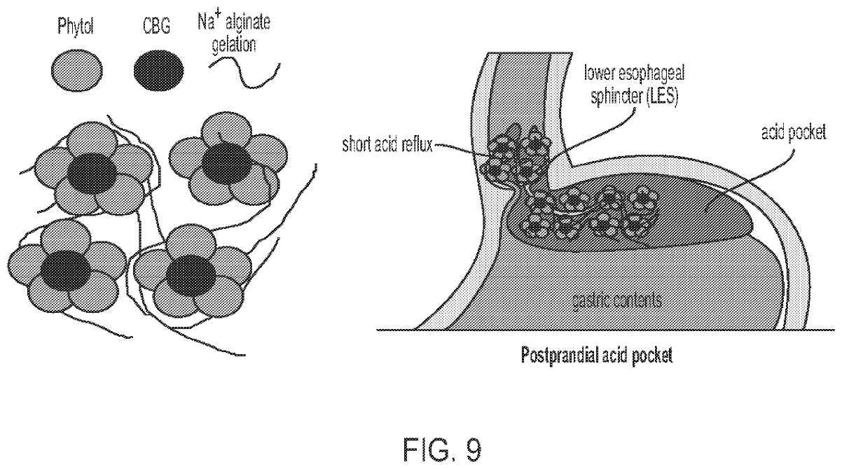

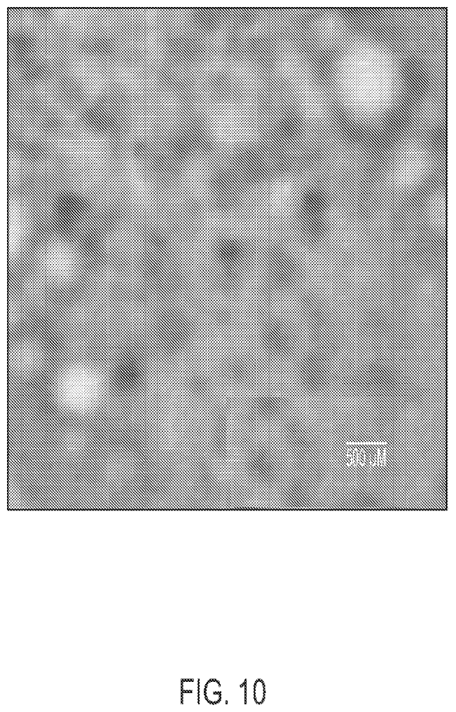

This example demonstrates the formulation of CBG-phytol sodium alginate. A drug loading of about 10% is achieved. Briefly, sodium alginate is dispersed into molecular grade water and pH is adjusted to 3.0 using dilute hydrochloric acid (1M). For amide bond formation, (3-Dimethylamino-propyl)-ethyl-carbodiimide Hydrochloride (EDC-HCl) is added until the solution is homogenous and then octylamine is added and maintained overnight at room temperature. The modified alginate is then combined with a cannabinoid/phytol solution suspended in ethanol (1 mg/ml CBG and 5 mg/ml phytol). The resulting solution appears miscible with polymer-forming particles evident containing the drug combination (FIG. 9). Hydrogel formation is then achieved by submersing the solution into a calcium chloride solution contained in a petri dish for 24 hours to ensure complete polymerization. Hydrogels are then dried overnight and kept at 4 until reconstituted for characterization and in-vivo experiments.

It will be apparent to those skilled in the art that various modifications and variations can be made without departing from the scope or spirit. Other embodiments will be apparent to those skilled in the art from consideration of the specification and practice disclosed herein. It is intended that the specification and examples be considered as exemplary only, with a true scope and spirit being indicated by the following claims.

1. A pharmaceutical composition comprising:
a) a cannabinoid represented by Formula (I): 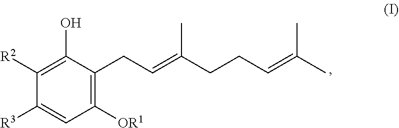
wherein R¹ is hydrogen or methyl; R² is hydrogen or —COOH; R³ is C3-C5 alkyl; or a salt thereof; and
b) a compound represented by Formula (II): 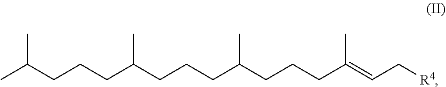
wherein R⁴ is halide or —OR⁵; wherein R⁵, when present, is hydrogen or —COR⁶; wherein R⁶, when present, is C1-C18 alkyl, or aryl; or
c) a terpene selected from (E)-β-caryophyllene, (Z)-β-caryophyllene, caryophyllene oxide, α-humulene, myrcene, limonene, linalool, or pinene;
wherein the cannabinoid and the compound represented by Formula (II), or the terpene, are present at a non-naturally occurring molar ratio; and
wherein the compound represented by Formula (II), or the terpene, is present at a molar amount that exceeds the molar amount of the cannabinoid.

2. The composition of claim 1, wherein the cannabinoid and the compound represented by Formula (II) or the terpene, are present at a molar ratio of about 1:5-1:10.

3. (canceled)

4. The composition of claim 1, wherein R³ is C₃H₇ or C₅H₁₁.

5. The composition of claim 1, wherein the cannabinoid is cannabigerovarin (CBGV), cannabigerovarinic acid (CBGVA), cannabigerol (CBG), cannabigerolic acid (CBVA), O-methylcannabigerol, or cannabigerolic acid methylether.

6. (canceled)

7. The composition of claim 1, which is substantially free of Δ⁹-tetrahydrocannabinol (Δ⁹-THC) and Δ⁸-tetrahydrocannabinol (Δ⁸-THC).

8. The composition of claim 1, which is substantially free of Δ⁹-tetrahydrocannabinol (Δ⁹-THC), Δ⁸-tetrahydrocannabinol (Δ⁸-THC), Δ⁸-tetrahydrocannabiphorol (Δ⁸-THCP), Δ⁹-tetrahydrocannabiphorol (Δ⁹-THCP), cannabichromene (CBC), cannabicyclol (CBL), cannabidiol (CBD), cannabidiphorol (CBDP), cannabielsoin (CBE), cannabinidiol (CBND), cannabinol (CBN), and cannabitriol (CBT).

9. The composition of claim 1, which is substantially free of any cannabinoid other than the cannabinoid represented by Formula (I).

10. The composition of claim 1, which consists essentially of the cannabinoid, and the compound represented by Formula (II) or the terpene.

11. The composition of claim 1, wherein the cannabinoid is present as an acceptable, non-naturally occurring salt.

12. The composition of claim 1, further comprising an acceptable, non-naturally occurring carrier.

13. The composition of claim 1, wherein the composition is in the form of an orally available solution, gel, or capsule.

14. A method of treating or preventing esophageal adenocarcinoma in a subject, the method comprising administering to the subject an effective amount of the composition of claim 1.

15. The method of claim 14, wherein the subject has or has been diagnosed with esophageal dysplasia, esophageal metaplasia, Barrett's Esophagus, gastroesophageal reflux disorder (GERD), or a condition associated with acid-biliary reflux.

16. The method of claim 14, comprising administering the composition orally.

17. (canceled)

18. (canceled)

19. (canceled)

20. (canceled)

21. (canceled)

22. (canceled)

23. (canceled)

24. A hydrogel comprising:
a) a polymer formed from an alginate and a hydrophobic amine;
b) a cannabinoid represented by Formula (I): 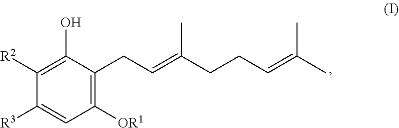
wherein R¹ is hydrogen or methyl; R² is hydrogen or —COOH; R³ is C3-C5 alkyl; or a salt thereof; and
c) a compound represented by Formula (II): 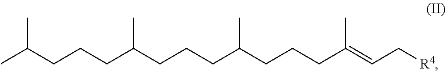
wherein R⁴ is halide or —OR⁵; wherein R⁵, when present, is hydrogen or —COR⁶; wherein R⁶, when present, is C1-C18 alkyl, or aryl; or
d) a terpene selected from (E)-β-caryophyllene, (Z)-β-caryophyllene, caryophyllene oxide, α-humulene, myrcene, limonene, linalool, or pinene;
wherein the cannabinoid and the compound represented by Formula (II), or the terpene, are present at a non-naturally occurring molar ratio; and
wherein the compound represented by Formula (II), or the terpene, is present at a molar amount that exceeds the molar amount of the cannabinoid.

25. The hydrogel of claim 24, wherein the alginate is sodium alginate.

26. The hydrogel of claim 24, wherein the hydrophobic amine is a C4-C12 alkyl amine.

27. The hydrogel of claim 24, wherein the hydrophobic amine is octyl amine.

28. The hydrogel of claim 24, wherein the cannabinoid is cannabigerol (CBG).

29. The hydrogel of claim 24, wherein the cannabinoid and the compound represented by Formula (II) or the terpene, are present at a molar ratio of about 1:5.

30-39. (canceled)
